(19) United States
(12) Patent Application Publication
(10) Pub. No.: US 2025/0186318 A1
(43) Pub. Date: Jun. 12, 2025

(54) Oral Care Compositions

(71) Applicant: Colgate-Palmolive Company, New York, NY (US)

(72) Inventors: Dailin CHEN, Guangzhou (CN); Xiaoyi HUANG, Guangzhou (CN); Shaopeng XU, Guangzhou (CN); Yun Po ZHANG, Kendall Park, NJ (US)

(73) Assignee: Colgate-Palmolive Company, New York, NY (US)

(21) Appl. No.: 18/973,959

(22) Filed: Dec. 9, 2024

(30) Foreign Application Priority Data
Dec. 7, 2023 (CN) 202311690447.1

(51) Int. Cl. A61K 8/365; A61K 8/27; A61K 8/34; A61Q 11/00

(52) U.S. Cl. CPC A61K 8/365; A61K 8/27; A61K 8/347; A61Q 11/00; A61K 2800/48; A61K 2800/592; A61K 2800/594

(57) ABSTRACT

Oral care compositions and, preferably, oral care compositions providing a reduction in gum bleeding are disclosed herein. In accordance with an aspect, provided is an oral care composition including oleanolic acid; eugenol; and a zinc source.

### CROSS-REFERENCE TO RELATED APPLICATIONS

This application claims the benefit of priority from Chinese Patent Application No. 202311690447.1, filed Dec. 7, 2023, titled Oral Care Compositions, which is hereby incorporated herein by reference in its entirety for all purposes.

### BACKGROUND

Gum bleeding is associated with many common oral conditions, such as gingivitis. Gum bleeding may be caused by a buildup of plaque, a soft, sticky, colorless film of bacteria that forms on the teeth and gums, and produces toxins that may inflame or infect the gum tissue to cause gingivitis. Gingivitis is the initial stage of gum disease and, if left untreated, may cause periodontitis.

Antibacterial agents have been used in oral care products to reduce plaque and gingivitis, and hence reduce gum bleeding. Similarly, hemostatic agents have also been used in oral care products to reduce gum bleeding. Oleanolic acid is believed to have both antibacterial and hemostatic properties, and there is a desire to create oral care compositions incorporating oleanolic acid. However, due to its hydrophobic properties, oleanolic acid is often dissolved in solvents, such as ethanol or dimethyl sulfoxide (DMSO), to facilitate its use. The use of such solvents limits the incorporation of oleanolic acid in oral care compositions which wish to avoid or limit the use of those ingredients.

Accordingly, it would be useful to develop new methods to incorporate oleanolic acid into oral care compositions, such as toothpastes and mouthwashes, and in particular, aqueous oleanolic acid solutions that can be used to prepare oral care compositions.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions and, preferably, oral care compositions providing a reduction in gum bleeding. In accordance with an aspect, provided is an oral care composition including oleanolic acid; eugenol; and a zinc source.

In accordance with another aspect, provided is an oral care composition including oleanolic acid; eugenol; a zinc source; and a basic amino acid.

In accordance with a further aspect, provided is provided is an oral care composition including oleanolic acid; eugenol, wherein the oleanolic acid and the eugenol are present in a weight ratio of the oleanolic acid to the eugenol of about 2:1 to about 3:1; and a zinc system comprising at least two zinc sources selected from zinc citrate, zinc oxide, and zinc phosphate.

A list of non-limiting embodiments of oral care compositions in accordance with aspects of the invention is provided below:

In accordance with an embodiment 1, provided is an oral care composition comprising: oleanolic acid; eugenol; and a zinc source.

In accordance with an embodiment 2, provided is the oral care composition according to claim 1, wherein the oleanolic acid and the eugenol are present in a weight ratio of the oleanolic acid to the eugenol of about 2:1 to about 3:1.

In accordance with an embodiment 3, provided is the oral care composition according to claim 1 or claim 2, wherein the oleanolic acid is present in an amount from about 0.01 to about 3 wt. %, based on the total weight of the oral care composition.

In accordance with an embodiment 4, provided is the oral care composition according to any foregoing claim, wherein the eugenol is present in an amount from about 0.01 to about 1.5 wt. %, based on the total weight of the oral care composition.

In accordance with an embodiment 5, provided is the oral care composition according to any foregoing claim, wherein the zinc source is selected from zinc citrate, zinc oxide, zinc phosphate, zinc lactate, zinc sulfate, zinc silicate, zinc gluconate, and a combination of two or more thereof.

In accordance with an embodiment 6, provided is the oral care composition according to any foregoing claim, wherein the zinc source comprises zinc citrate, zinc oxide, zinc phosphate, zinc lactate, or a combination of two or more thereof.

In accordance with an embodiment 7, provided is the oral care composition according to any foregoing claim, wherein the oral care composition comprises a zinc system comprising at least two zinc sources selected from zinc citrate, zinc oxide, and zinc phosphate.

In accordance with an embodiment 8, provided is the oral care composition according to any foregoing claim further comprising: a basic amino acid, optionally, in an amount from about 0.1 to about 10 wt. %, based on the total weight of the oral care composition.

In accordance with an embodiment 9, provided is the oral care composition according to claim 8, wherein the basic amino acid comprises arginine, lysine, histidine, or a combination thereof.

In accordance with an embodiment 10, provided is the oral care composition according to any foregoing claim further comprising: one or more polyol, optionally in an amount from about 15 to about 50 wt. %, based on the total weight of the oral care composition.

In accordance with an embodiment 11, provided is the oral care composition according to claim 10, wherein the polyol comprises glycerin, inositol, maltitol, mannitol, sorbitol, xylitol, propylene glycol, polypropylene glycol (PPG), polyethylene glycol (PEG), a block copolymer of PPG and PEG, a saccharide, or a combination of two or more thereof.

In accordance with an embodiment 12, provided is the oral care composition according to one of claim 10 to claim 11, wherein the one or more polyol comprises a polyol system.

In accordance with an embodiment 13, provided is the oral care composition according to claim 12, wherein the polyol system comprises glycerin and sorbitol.

In accordance with an embodiment 14, provided is the oral care composition according to claim 13, wherein the glycerin and sorbitol are present in a weight ratio of glycerin to sorbitol of about 1:1 to about 3:1.

In accordance with an embodiment 15, provided is the oral care composition according to any foregoing claim further comprising: an abrasive, optionally, in an amount from about 1 to about 35 wt. %, based on the total weight of the oral care composition.

In accordance with an embodiment 16, provided is the oral care composition according to claim 15, wherein the abrasive is selected from silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof.

In accordance with an embodiment 17, provided is the oral care composition according to claim 15, wherein the abrasive consists of silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof.

In accordance with an embodiment 18, provided is the oral care composition according to one of claim 15 to claim 17, wherein the abrasive comprises silica.

In accordance with an embodiment 19, provided is the oral care composition according to any foregoing claim further comprising: a fluoride source, optionally, in an amount from about 0.1 to about 7 wt. %, based on the total weight of the oral care composition.

In accordance with an embodiment 20, provided is the oral care composition according to claim 19, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

In accordance with an embodiment 21, provided is the oral care composition according to any foregoing claim further comprising: an anti-calculus agent in an amount from about 0.5 to about 10 wt. %, based on the total weight of the oral care composition.

In accordance with an embodiment 22, provided is the oral care composition according to claim 21, wherein the anti-calculus agent is selected from tetrasodium pyrophosphate, ammonium tripolyphosphate, hexametaphosphate salt, polycarboxylate polymers, polyvinyl methyl ether/maleic anhydride (PVME/MA) copolymers, and a combination of two or more thereof.

In accordance with an embodiment 23, provided is the oral care composition according to any foregoing claim further comprising: a thickening agent, optionally present in an amount about 1 to about 8 wt. %, based on the total weight of the oral care composition.

In accordance with an embodiment 24, provided is the oral care composition according to claim 23, wherein the thickening system comprises a thickening silica.

In accordance with an embodiment 25, provided is an oral care composition comprising: oleanolic acid; eugenol; a zinc source; and a basic amino acid.

In accordance with an embodiment 26, provided is the oral care composition according to claim 25, wherein the basic amino acid comprises arginine, lysine, or a combination thereof.

In accordance with an embodiment 27, provided is an oral care composition comprising: oleanolic acid; eugenol, wherein the oleanolic acid and the eugenol are present in a weight ratio of the oleanolic acid to the eugenol of about 2:1 to about 3:1; and a zinc system comprising at least two zinc sources selected from zinc citrate, zinc oxide, and zinc phosphate.

### BRIEF DESCRIPTION OF THE DRAWINGS

The present invention may become more fully understood from the detailed description and the accompanying drawings, wherein like elements are labeled similarly and in which:

FIG. 1 is a bar graph of the platelet area increasing ratio of a non-limiting, example composition as compared to a comparative composition according to aspects of the invention; and

FIG. 2 is a bar graph of the velocity decrease of a non-limiting, example composition as compared to a comparative composition according to aspects of the invention.

It should be understood that the various aspects are not limited to the arrangements and instrumentality shown in the drawings.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “∘” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a polyol and a humectant. If a particular oral care composition recites both a polyol and a humectant, glycerin will serve only as either a polyol or a humectant—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions and, preferably, oral care compositions providing a reduction in gum bleeding. In accordance with an aspect, provided is an oral care composition including oleanolic acid; eugenol; and a zinc source.

In accordance with another aspect, provided is an oral care composition including oleanolic acid; eugenol; a zinc source; and a basic amino acid.

In accordance with a further aspect, provided is provided is an oral care composition including oleanolic acid; eugenol, wherein the oleanolic acid and the eugenol are present in a weight ratio of the oleanolic acid to the eugenol of about 2:1 to about 3:1; and a zinc system comprising at least two zinc sources selected from zinc citrate, zinc oxide, and zinc phosphate.

The oral care compositions may be in the form of dentifrice, toothpaste, mouthwash, gels, varnishes, leave-on products (e.g., in the form of a gel, varnish, etc.), and/or the like. Suitable components, such as those listed below, may be included or excluded from the formulations for the oral care compositions depending on the specific combination of other ingredients and the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions may be in the form of dentifrice, toothpaste, mouthwash, gels, varnishes, leave-on products (e.g., in the form of a gel, varnish, etc.), and/or the like. Suitable components, such as those listed below, may be included or excluded from the formulations for the oral care compositions depending on the specific combination of other ingredients and the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions typically comprise oleanolic acid, eugenol, and one or more zinc source in certain amounts and ratios thereof. The oleanolic acid may be incorporated into the oral care composition in the form of a salt. The amount of oleanolic acid and/or a salt thereof in the oral care composition may be from about 0.01 to about 3 wt. %, based on the total weight of the oral care composition. For instance, the oral care composition may include oleanolic acid and/or a salt thereof, preferably, in an amount from about 0.01 to about 3 wt. %, about 0.01 to about 2 wt. %, about 0.01 to about 1 wt. %, about 0.01 to about 0.5 wt. %, about 0.01 to about 0.3 wt. %, about 0.01 to about 0.2 wt. %, about 0.01 to about 0.1 wt. %; from about 0.05 to about 3 wt. %, about 0.05 to about 2 wt. %, about 0.05 to about 1 wt. %, about 0.05 to about 0.5 wt. %, about 0.05 to about 0.3 wt. %, about 0.05 to about 0.2 wt. %, about 0.05 to about 0.1 wt. %; from about 0.1 to about 3 wt. %, about 0.1 to about 2 wt. %, about 0.1 to about 1 wt. %, about 0.1 to about 0.5 wt. %, about 0.1 to about 0.3 wt. %, about 0.1 to about 0.2 wt. %; from about 0.2 to about 3 wt. %, about 0.2 to about 2 wt. %, about 0.2 to about 1 wt. %, about 0.2 to about 0.5 wt. %, about 0.2 to about 0.3 wt. %; from about 0.5 to about 3 wt. %, about 0.5 to about 2 wt. %, about 0.5 to about 1 wt. %; from about 1 to about 3 wt. %, about 1 to about 2 wt. %, or any range or subrange thereof, based on the total weight of the oral care composition. The oleanolic acid may be obtained from certain medicinal plants, such as Calendula officinalis L. (marigold), Ligustrum lucidum Ait (oleaceae), and Hemsleya chinensis Cogn. The following is the chemical structure of oleanic acid:

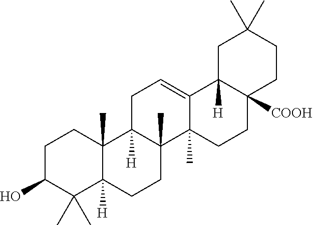

Eugenol is typically present in the oral care compositions. The amount of eugenol may be present in an amount from about 0.01 to about 1.5 wt. %, based on the total weight of the oral care composition. For instance, the oral care composition may include eugenol in an amount, preferably, from about 0.01 to about 1.5 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %; from about 0.05 to about 1.5 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 1.5 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.2 to about 1.5 wt. %, about 0.2 to about 1 wt. %, about 0.2 to about 0.8 wt. %, about 0.2 to about 0.6 wt. %, about 0.2 to about 0.4 wt. %; from about 0.5 to about 1.5 wt. %, about 0.5 to about 1 wt. %, about 0.5 to about 0.8 wt. %, about 1 to about 1.5 wt. %, or any range or subrange formed therefrom, based on the total weight of the oral care composition.

Eugenol (4-allyl-2-methoxyphenol), is a naturally occurring phenol essential oil extracted from, e.g., cloves, nutmeg, cinnamon, basil, and bay leaf. Eugenol may also be provided as methyl eugenol or iso-eugenol. For instance, the oral care composition may comprise methyl eugenol, iso-eugenol, or a combination thereof.

The oral care composition may, in some preferred embodiments, be specifically formulated to have certain weight ratios of the oleanolic acid to the eugenol. For instance, the oral care composition may preferably have a weight ratio of the oleanolic acid to the eugenol of about 1:1 to about 3:1. In some preferred embodiments, the oral care composition has a weight ratio of the oleanolic acid to the eugenol of about 1.5:1 to about 3:1, about 2:1 to about 3:1, about 2.5:1 to about 3:1, about 1:1 to about 2.5:1, about 1.5:1 to about 2.5:1, about 2:1 to about 2.5:1, about 1:1 to about 2:1, about 1.5:1 to about 2:1, or any range or subrange thereof.

The oral care compositions also typically include a zinc source. The zinc source may be a zinc ion source. The zinc source may be present in the oral care composition in an amount from about 0.1 to about 8 wt. %, based on the total weight of the oral care composition. For example, the amount of zinc source present in the oral care composition may be from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 8 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

The zinc source may be in the form of a salt. For example, the zinc source may comprise one or more zinc salt(s) chosen from zinc sulfate, zinc chloride, zinc acetate, zinc phenol sulfonate, zinc borate, zinc bromide, zinc nitrate, zinc glycerophosphate, zinc benzoate, zinc carbonate, zinc carnosine, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, zinc lactate, zinc glycinate, zinc phosphate, and/or a combination of two or more thereof. In some embodiments, the zinc source is selected from zinc citrate, zinc oxide, zinc phosphate, zinc lactate, zinc sulfate, zinc silicate, zinc gluconate, and a combination of two or more thereof. The zinc source may comprise zinc citrate, zinc oxide, zinc phosphate, zinc lactate, or a combination of two or more thereof. In at least one embodiment, the zinc source consists of one or more of zinc citrate, zinc oxide, zinc phosphate, zinc lactate, or a combination of two or more thereof.

The oral care composition may include two or more, three or more, four or more, five or more, or six or more zinc sources. For instance, oral care composition may comprise 2 to 7, 2 to 6, 2 to 5, 2 to 5, or 2 to 4; 3 to 7, 3 to 6, 2 to 5, or 3 to 5 zinc sources. In some embodiments, the oral care composition comprises zinc oxide, zinc citrate, zinc phosphate, or a combination of two or more thereof. For example, the zinc system may comprise at least two zinc sources, such as those selected from zinc citrate, zinc oxide, zinc phosphate, and a combination thereof.

The oral care compositions may be formulated to have a weight ratio of the amount of zinc oxide to zinc citrate of from about 1.5:1 to about 4.5:1. For instance, the weight ratio of the amount of zinc oxide to zinc citrate may be from about 1.5:1 to about 4.5:1, about 1.5:1 to about 4:1, about 1.5:1 to about 3.5:1, about 1.5:1 to about 3:1, about 1.5:1 to about 2.5:1; from about 2:1 to about 4.5:1, about 2:1 to about 4:1, about 2:1 to about 3.5:1, about 2:1 to about 3:1; from about 2.5:1 to about 4.5:1, about 2.5:1 to about 4:1, about 2.5:1 to about 3.5:1; from about 3:1 to about 4.5:1, about 3:1 to about 4:1, or any range or subrange formed thereof. In some embodiments, the weight ratio of the amount of zinc oxide to zinc citrate is about 2:1, about 2.5:1, about 3:1, about 3.5:1, about 4:1, or a range formed thereof.

The oral care composition may comprise one or more basic amino acid, preferably in an effective amount. In some embodiments, the one or more amino acid(s) may be present in the oral care composition in an amount from about 0.1 to about 10 wt. %, about 0.1 to about 8 wt. %, about 0.1 to about 6 wt. %, about 0.1 to about 4 wt. %, about 0.1 to about 2 wt. %, about 0.1 to about 1 wt. %, about 0.1 to about 0.5 wt. %; from about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 10 wt. %, about 1 to about 8 wt. %, about 1 to about 6 wt. %, about 1 to about 4 wt. %, about 1 to about 2 wt. %; from about 2 to about 10 wt. %, about 2 to about 8 wt. %, about 2 to about 6 wt. %, about 2 to about 4 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %; from about 6 to about 10 wt. %, about 6 to about 8 wt. %; from about 8 to about 10 wt. %, including any range or subrange thereof, based on the total weight of the oral care composition.

The one or more basic amino acids may be selected from naturally occurring basic amino acids, such as arginine, lysine, and histidine, and non-naturally occurring basic amino acids having a carboxyl group and an amino group in the molecule, which are water-soluble and provide an aqueous solution with a pH of 7 or greater. The one or more basic amino acid may be selected from lysine, arginine, citrulline, ornithine, and a combination of two or more thereof. For instance, the oral care composition may comprise one or more of L-lysine, L-arginine, D-lysine, D-glycine, or a combination of two or more thereof. In at least one preferred embodiment, the basic amino acid is selected from L-glycine, L-arginine, and a combination thereof. In some embodiments, the only amino acids in the oral care composition are basic amino acids, such as those disclosed herein.

Examples of additional amino acids that may be incorporated or excluded from the oral care composition include glycine, serine, citrulline, ornithine, creatine, histidine, diaminobutanoic acid, diaminoproprionic acid, salts thereof or combinations thereof. Additionally or alternatively, the oral care composition may include a neutral amino acids, such as those selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof. In some embodiments, however, the oral care composition is substantially free of or free of neutral amino acids.

The oral care compositions may include one or more polyol(s). The one or more polyol(s) may be present in the oral care composition in an amount from about 15 to about 50 wt. %, based on the total weight of the oral care composition. For example, the total amount of polyol(s) present in the oral care composition may be from about 15 to about 45 wt. %, about 15 to about 37 wt. %, about 15 to about 31 wt. %, about 5 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 58 wt. %, about 37 to about 54 wt. %, about 37 to about 50 wt. %, about 37 to about 45 wt. %; from about 40 to about 50 wt. %, about 40 to about 45 wt. %; from about 43 to about 50 wt. %, about 43 to about 48 wt. %; from about 45 to about 50 wt. %, about 45 to about 48 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). In some preferred embodiments, the polyol(s) comprises glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof. The polyol may be a humectant including, e.g., glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer, and a combination of two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, dipropylene glycol, or a combination of two or more thereof. Additional, non-limiting examples of polyols that may, optionally, be included in or exclude from the oral care include and/or may be chosen from: alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and/or a mixture of two or more thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500, or any range or subrange thereof. In some embodiments, the polyol is a polyethylene glycol, such as polyethylene glycol 600. In some embodiments, the oral care composition comprises one or more polyethylene glycols, e.g., polyethylene glycols in a molecular weight range from 200 to 800. For example, the oral care compositions may comprise one or more of polyethylene glycol 200, polyethylene glycol 300, polyethylene glycol 400, polyethylene glycol, 600 or polyethylene glycol 800.

In some cases, the oral care composition includes a polyol system comprising a plurality of polyols. For example, the polyol system may comprise two or more polyols. In some instances, at least one of the polyols is selected from glycerin and sorbitol. In at least one embodiment, the oral care composition comprises glycerin and sorbitol. The oral care compositions may, in some preferred embodiments, have a polyol system comprising glycerin and sorbitol present in a weight ratio of glycerin to sorbitol of about 1:1 to about 3:1. For instance, the oral care composition may have a weight ratio of glycerin to sorbitol of about 1:1 to about 3:1, about 1:1 to about 2.75:1, about 1:1 to about 2.5:1, about 1:1 to about 2.25:1, about 1:1 to about 2:1; from about 1:1 to about 3:1, about 1:1 to about 2.75:1, about 1:1 to about 2.5:1, about 1:1 to about 2.25:1, about 1:1 to about 2:1; from about 1.25:1 to about 3:1, about 1.25:1 to about 2.75:1, about 1.25:1 to about 2.5:1, about 1.25:1 to about 2.25:1, about 1.25:1 to about 2:1; from about 1.5:1 to about 3:1, about 1.5:1 to about 2.75:1, about 1.5:1 to about 2.5:1, about 1.5:1 to about 2.25:1, about 1.5:1 to about 2:1; from about 1.75:1 to about 3:1, about 1.75:1 to about 2.75:1, about 1.75:1 to about 2.5:1, about 1.75:1 to about 2.25:1, about 1.75:1 to about 2:1, or a range or subrange thereof.

The oral care compositions may include one or more abrasive(s), e.g., in an amount from about 1 to about 35 wt. %, based on the total weight of the oral care composition. In some embodiments, the one or more abrasive(s) is present in an amount from about 1 to about 30 wt. %, about 1 to about 25 wt. %, about 1 to about 21 wt. %, about 1 to about 17 wt. %, about 1 to about 14 wt. %, about 1 to about 11 wt. %; from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the oral care composition.

The one or more abrasive(s) may include: silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, and urea-formaldehyde resin, polycarbonate, silicon carbide, boron carbide, microcrystalline wax, microcrystalline cellulose, including combinations of colloidal microcrystalline cellulose and carboxymethylcellulose, and combinations and derivatives thereof. For example, the one or more abrasive may be selected from silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, resinous abrasive materials such as particulate condensation products of urea and formaldehyde, and combinations thereof.

As used herein, “mica” refers to any of a group of hydrous aluminum silicate minerals with plate morphology and/or perfect basal (micaceous) cleavage. Mica can be, for example, sheet mica, scrap mica or flake mica, as exemplified by muscovite, biotite or phlogopite type micas. The abrasive may be selected from insoluble phosphates, such as as orthophosphates, polymetaphosphates, pyrophosphates, and a combination thereof. Synthetic silicas include both silica gels and precipitated silicas that are prepared by the neutralization of aqueous silicate solutions with a strong mineral acid. Abrasives comprising silica may be useful in certain embodiments of the oral care composition. In certain embodiments, the oral compositions may comprise a particularly efficacious combination of silica abrasive particle species. For example, the abrasive(s) be selected from high cleaning silica.

The oral care compositions may include an abrasive system comprising two or more abrasives. For example, an abrasive system may comprise 2 to 7, 2 to 6, 2 to 5, 2 to 5, or 2 to 4; 3 to 7, 3 to 6, 2 to 5, or 3 to 5 abrasives. The abrasives may comprise one or more cleaning abrasive and/or one or more polishing abrasives. As appreciated by one of skill in the art, a single abrasive species typically performs at least some cleaning and polishing simultaneously. However, particles are generally categorized in the art by the predominant effect they have on a target oral surface. Typically, “polishing abrasives” are considered to be relatively small particles having high hardness, where abrasives with relatively large particle sizes and low hardness are considered to be “cleaning abrasives.” In certain embodiments, the oral care composition comprises two or more abrasives comprising silica. In some embodiments, the first abrasive is selected to be a harder and smaller abrasive, e.g., a higher cleaning and/or polishing abrasive, and the second abrasive is a typical cleaning abrasive. In some embodiments, the oral care composition includes at least one polishing abrasive and/or at least one cleaning abrasive. Further examples of abrasives are disclosed in U.S. Patent Publication No. 2007/140986, which is incorporated herein in its entirety by reference for all purposes.

In some cases, the abrasive is selected from silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof. The oral care composition may have abrasive(s) consisting of silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof in certain embodiments. In at least one embodiment, the abrasive is silica.

The oral care compositions may include a fluoride ion source. The fluoride ion source may be present in an effective amount. In some cases, the fluoride ion source is present in the oral care composition in an amount of about 0.01 to about 5 wt. %, based on the total weight of the oral care composition. For example, the fluoride ion source is present in an amount of about 0.01 to about 5 wt. %, about 0.01 to about 4 wt. %, about 0.01 to about 3 wt. %, about 0.01 to about 2 wt. %, about 0.01 to about 1 wt. %; from about 0.05 to about 5 wt. %, about 0.05 to about 4 wt. %, about 0.05 to about 3 wt. %, about 0.05 to about 2 wt. %, about 0.05 to about 1 wt. %; from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion source are disclosed in U.S. Pat. Nos. 3,535,421, 4,885,155, and 3,678,154, the disclosure of each of which is hereby incorporated by reference in their entirety.

In certain embodiments, the oral care composition of the disclosure may contain a source of fluoride ions or fluorine-providing agents in amounts sufficient to deliver, in total, from 25 ppm to 25,000 ppm (mass fraction) of fluoride ions, generally at least 500 ppm, e.g., from about 500 to about 2000 ppm, from about 800 to about 1800 ppm, from about 1000 to about 1600 ppm, from about 1200 to about 1550 ppm, or about 1450 ppm.

The oral care compositions may include an anti-calculus agent. The amount of anti-calculus agent(s) present in the oral care composition may be from about 0.5 to about 10 wt. %, based on the total weight of the oral care composition. In some instances, the anti-calculus agent(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anti-calculus agent(s) may be chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. Examples of anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, e.g., as tetrasodium pyrophosphate.

Additionally or alternatively, the oral care composition may comprise one or more thickening agent(s). The total amount of thickening agent(s) in the oral care composition may be from about 1 to about 8 wt. %, about 1 to about 7 wt. %, about 1 to about 6 wt. %, about 1 to about 5 wt. %, about 1 to about 4 wt. %; about 2 to about 8 wt. %, about 2 to about 7 wt. %, about 2 to about 6 wt. %, about 2 to about 5 wt. %, about 2 to about 4 wt. %; from about 3 to about 10 wt. %, about 3 to about 8 wt. %, about 3 to about 7 wt. %, about 3 to about 6 wt. %, about 3 to about 5 wt. %, about 3 to about 4 wt. %; from about 3.5 to about 10 wt. %, about 3.5 to about 8 wt. %, about 3.5 to about 7 wt. %, about 3.5 to about 6 wt. %, about 3.5 to about 5 wt. %, about 3.5 to about 4 wt. %; from about 4 to about 10 wt. %, about 4 to about 8 wt. %, about 4 to about 7 wt. %, about 4 to about 6 wt. %; from about 5 to about 10 wt. %, about 5 to about 8 wt. %, about 5 to about 7 wt. %; from about 6 to about 10 wt. %, about 6 to about 8 wt. %, or any range or subrange thereof, based on the total weight of the oral care composition.

Thickening agent(s) provide a desirable consistency or are used to stabilize and/or enhance the solubility of other ingredients. In some embodiments, the viscosity of the oral care composition is from about 10,000 centipoise (cPs) to about 500,000 cPs at a temperature of 25° C. For example, the viscosity of the oral care composition is from about 50,000 cPs to about 400,000 cPs at a temperature of 25° C. In one embodiment, the viscosity of the oral care composition is from about 125,000 cPs to about 300,000 cPs at a temperature of 25° C.

Examples of thickening agents that may be included or in some cases excluded from the oral care composition include carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose. Natural gums, such as karaya, gum arabic, and gum tragacanth can also be incorporated. Colloidal magnesium aluminum silicate or finely divided silica can be used as thickening agent to further improve the composition's texture. In some embodiments, the thickening agent may include polyethylene glycols, and polysaccharides (e.g., cellulose derivatives, for example carboxymethyl cellulose, or polysaccharide gums, for example xanthan gum or carrageenan gum). In certain embodiments, thickening agents may be selected from carboxyvinyl polymers, carrageenan, xanthan gum, hydroxyethyl cellulose and water soluble salts of cellulose ethers, such as sodium carboxymethyl cellulose and sodium carboxymethyl hydroxyethyl cellulose, and mixtures thereof. In certain embodiments, the acidic polymers (e.g., carboxymethyl cellulose) may be provided in the form of their free acids or partially or fully neutralized water soluble alkali metal (e.g., potassium and sodium) or ammonium salts. In certain embodiments, the oral care composition comprises a polymer selected from carboxymethyl cellulose (free form or a salt, e.g., sodium salt), a gum (e.g., xanthan gum, carrageenan gum, or gum arabic), polyethylene glycol (e.g., polyethylene glycol 200, 400, 600 or 800), or a mixture thereof. In certain embodiments, the thickening agent is carboxymethylcellulose or a salt derivative thereof. In certain embodiments, the oral care composition comprises carboxymethylcellulose, a silica thickener, or a combination thereof. The oral care compositions may include a thickening agent that is selected from silica thickeners. Non-limiting examples of a silica thickener include silica dioxide.

The oral care composition may include optionally include one or more whitening agent(s). As used herein, a “whitening agent” is a material that affects whitening of a tooth surface to which it is applied. For example, in some embodiments, the whitening agent is an oxidizing agent. In its broadest sense, “oxidizing agent” is intended to include those compounds which can accept an electron from another molecule in the environment of the oral cavity without having a deleterious or unacceptably harmful effect on the oral cavity in normal and accepted use.

In some embodiments, the whitening agent may include peroxides and hydroperoxides, such as hydrogen peroxide, peroxides of alkali and alkaline earth metals, organic peroxy compounds, peroxy acids, salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include urea peroxide, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids, such as alkyl peroxy acids, monoperoxyphthalate and mixtures thereof, as well as inorganic peroxy acid salts, such as percarbonate, perphosphate, perborate and persilicate salts of alkali and alkaline earth metals such as lithium, potassium, sodium, magnesium, calcium and barium, and mixtures thereof. In some embodiments a non-peroxide whitening agent may be provided. Examples of non-peroxy compounds include chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite.

In some embodiments, the whitening agent may be present in an amount from about 1 to about 35 wt. %, based on a total weight of the oral care composition. For example, the oral care may include the one or more whitening agent(s) in an amount from about 1 to about 30 wt. %, about 1 to about 25 wt. %, about 1 to about 21 wt. %, about 1 to about 17 wt. %, about 1 to about 14 wt. %, about 1 to about 11 wt. %; from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the oral care composition.

In some embodiments, however, the oral care composition may be substantially free of or free of one or more of the whitening agent described above. In some cases, the oral care composition is substantially free or free of whitening agent(s).

The oral care compositions may include one or more surfactant. The total amount of surfactant(s) in the oral care composition may be from about 1 to about 8 wt. %, about 1 to about 7 wt. %, about 1 to about 6 wt. %, about 1 to about 5 wt. %, about 1 to about 4 wt. %; about 2 to about 8 wt. %, about 2 to about 7 wt. %, about 2 to about 6 wt. %, about 2 to about 5 wt. %, about 2 to about 4 wt. %; from about 3 to about 10 wt. %, about 3 to about 8 wt. %, about 3 to about 7 wt. %, about 3 to about 6 wt. %, about 3 to about 5 wt. %, about 3 to about 4 wt. %; from about 3.5 to about 10 wt. %, about 3.5 to about 8 wt. %, about 3.5 to about 7 wt. %, about 3.5 to about 6 wt. %, about 3.5 to about 5 wt. %, about 3.5 to about 4 wt. %; from about 4 to about 10 wt. %, about 4 to about 8 wt. %, about 4 to about 7 wt. %, about 4 to about 6 wt. %; from about 5 to about 10 wt. %, about 5 to about 8 wt. %, about 5 to about 7 wt. %; from about 6 to about 10 wt. %, about 6 to about 8 wt. %, or any range or subrange thereof, based on the total weight of the oral care composition.

The one or more surfactant(s) may comprise an anionic surfactant, such as those selected from water-soluble, water-miscible salts of alkyl sulfate, such as those having from 8 to 20 carbon atoms in the alkyl radical (e.g., sodium alkyl sulfate) and the water-soluble or water-miscible salts of sulfonated monoglycerides of fatty acids having from 8 to 20 carbon atoms. Examples of anionic surfactants, which may be included or excluded in some instances from the oral care composition, include sodium lauryl sulfate, sodium lauryl sarcosinate, sodium cocoyl methyl taurate, sodium monoglyceride sulfate, sodium cetearyl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and combinations thereof. In one embodiment, sodium lauryl sulfate is a preferred surfactant. Additional anionic surfactants are disclosed in U.S. Pat. No. 3,959,458, which is incorporated herein in its entirety for all purposes.

Further anionic surfactants of mention include long chain alkyl (C₆-C₂₂) materials, such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, e.g., ethoxylated, although alkoxylation is not required.

The oral care composition may include a surfactant other than an anionic surfactant, such as a nonionic surfactant, amphoteric surfactant, or cationic surfactant. Examples of amphoteric surfactants include betaine surfactants, such as cocamidopropyl betaine, lauryl dimethyl betaine (sold under the trade name Macat LB), cetyl dimethyl betaine, and cocoamphodiacetate. Examples of nonionic surfactants, which may be included or excluded from the oral care composition, include glucosides, compounds produced by the condensation of alkylene oxide groups (hydrophilic in nature) with an organic hydrophobic compound, which may be aliphatic or alkylaromatic in nature.

The oral care composition may comprise water in an amount from about 10 to about 45 wt. %, based on the total weight of the oral care composition. In some embodiments, the oral care composition comprises water in an amount of about 10 to about 45 wt. %, about 10 to about 40 wt. %, about 10 to about 35 wt. %, about 10 to about 32 wt. %, about 10 to about 29 wt. %, about 10 to about 26 wt. %, about 10 to about 23 wt. %, about 10 to about 17 wt. %; from about 15 to about 45 wt. %, about 15 to about 40 wt. %, about 15 to about 35 wt. %, about 15 to about 32 wt. %, about 15 to about 29 wt. %, about 15 to about 26 wt. %, about 15 to about 23 wt. %, about 15 to about 19 wt. %; from about 19 to about 45 wt. %, about 19 to about 40 wt. %, about 19 to about 35 wt. %, about 19 to about 32 wt. %, about 19 to about 29 wt. %, about 19 to about 26 wt. %, about 19 to about 23 wt. %; from about 22 to about 45 wt. %, about 22 to about 40 wt. %, about 22 to about 35 wt. %, about 22 to about 32 wt. %, about 22 to about 29 wt. %, about 22 to about 26 wt. %; from about 25 to about 45 wt. %, about 25 to about 40 wt. %, about 25 to about 35 wt. %, about 25 to about 32 wt. %, about 25 to about 29 wt. %; from about 28 to about 45 wt. %, about 28 to about 40 wt. %, about 28 to about 35 wt. %, about 28 to about 32 wt. %; from about 31 to about 45 wt. %, about 31 to about 40 wt. %, about 31 to about 35 wt. %; from about 34 to about 45 wt. %, about 34 to about 40 wt. %, about 37 to about 45 wt. %, or any range or subrange thereof, based on the total weight of the oral care composition.

The oral care compositions may include one or more sweeteners. Examples of sweeteners include caloric sweeteners and/or non-caloric sweeteners. Non-caloric sweeteners that are worth mentioning include saccharin, for example, sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 50 wt. %, based on the total weight of the oral care composition. For example, the oral care composition may have a total amount of sweetener(s) from about 0.1 to about 40 wt. %, about 0.1 to about 30 wt. %, about 0.1 to about 20 wt. %, about 0.1 to about 10 wt. %, about 0.1 to about 5 wt. %, about 0.1 to about 3 wt. %; from about 1 to about 50 wt. %, about 1 to about 40 wt. %, about 1 to about 30 wt. %, about 1 to about 20 wt. %, about 1 to about 10 wt. %, about 1 to about 5 wt. %, about 1 to about 3 wt. %; from about 5 to about 50 wt. %, about 5 to about 40 wt. %, about 5 to about 30 wt. %, about 5 to about 20 wt. %, about 5 to about 10 wt. %; from about 10 to about 50 wt. %, about 10 to about 45 wt. %, about 10 to about 40 wt. %, about 10 to about 35 wt. %, about 10 to about 30 wt. %, about 10 to about 25 wt. %; from about 15 to about 50 wt. %, about 15 to about 45 wt. %, about 15 to about 40 wt. %, about 15 to about 37 wt. %, about 15 to about 34 wt. %, about 15 to about 31 wt. %, about 15 to about 28 wt. %, about 15 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 50 wt. %, about 37 to about 45 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less of caloric sweeteners, based on the weight of the oral care composition. In at least one embodiment, the oral care composition contains about 0 wt. % or 0 wt. % of caloric sweeteners, based on the weight of the oral care composition.

The oral care compositions may include a flavoring agent. The flavoring agent is typically incorporated in the oral care composition at a concentration of about 0.01 to about 3 wt. %, by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, and various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more colorants and/or pigments. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. Non-limiting examples of pigments include titanium dioxide, zinc oxide, kaolin, mica etc. The concentrations of the colorant(s) and/or the pigment(s) in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

The oral care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween, e.g., as measured as a 10 wt. % solution. The pH of the solution (e.g., any of the foregoing pH ranges) may be measured as a 10 wt. % solution, whereby the solution comprises 10 wt. % of the oral care composition with the remainder being water.

### EXAMPLES

### Example 1

A non-limiting, example oral care composition (Example Composition A) was prepared in the form of a dentifrice according to aspects of the invention. A comparative composition (Comparative Composition 1) was also prepared in the form of a dentifrice. The formulations of Example Composition A and Comparative Composition 1 are shown in Table 1.

TABLE 1

| | Example | Comparative |
|---|---|---|
| | Composition A | Composition 1 |
| US INCI Compound Name | (wt. %) | (wt. %) |
| Zinc Citrate | 2 | 2 |
| Oleanolic Acid | 0.1 | |
| Eugenol | 0.05 | |
| Sorbitol | 15.1 | 15.1 |
| Glycerin | 11.4 | 11.4 |
| Polyethylene Glycol 600 | 3 | 3 |
| Abrasive Silica | 20 | 20 |
| Thickening Silica and | 2.2 | 2.2 |
| Xanthan Gum | | |
| Sodium Lauryl Sulfate | 2.3 | 2.3 |
| Tetrapotassium | 2.4 | 2.4 |
| Pyrophosphate | | |
| Sodium CMC | 0.7 | 0.7 |
| Maleic Anhydride Polymers | 1.5 | 1.5 |
| and Copolymers | | |
| Sodium | 1.1 | 1.1 |
| Monofluorophosphate | | |
| Flavoring and Saccharin | 1.8 | 1.8 |
| Benzyl Alcohol | 0.2-0.5 | 0.2-0.5 |
| Sodium Hydroxide | 0.3-1 | 0.3-1 |
| Titanium Dioxide | 0.1-0.7 | 0.1-0.7 |
| Water | QS to 100 | QS to 100 |

### Example 2

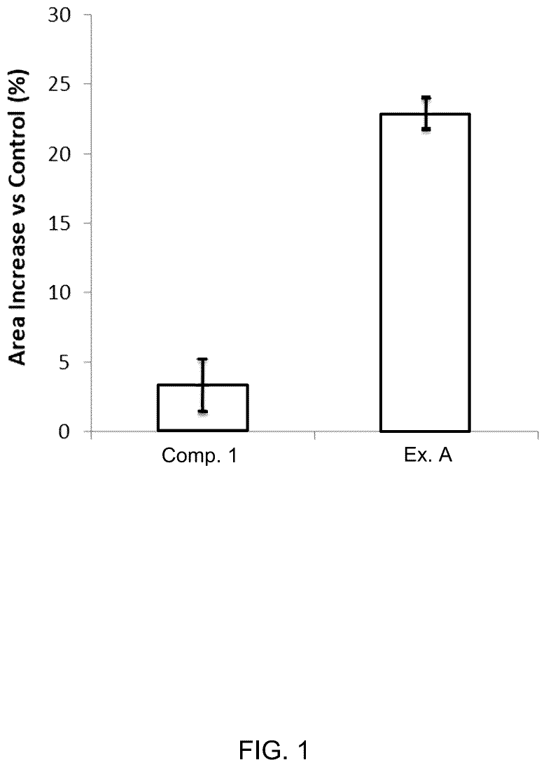

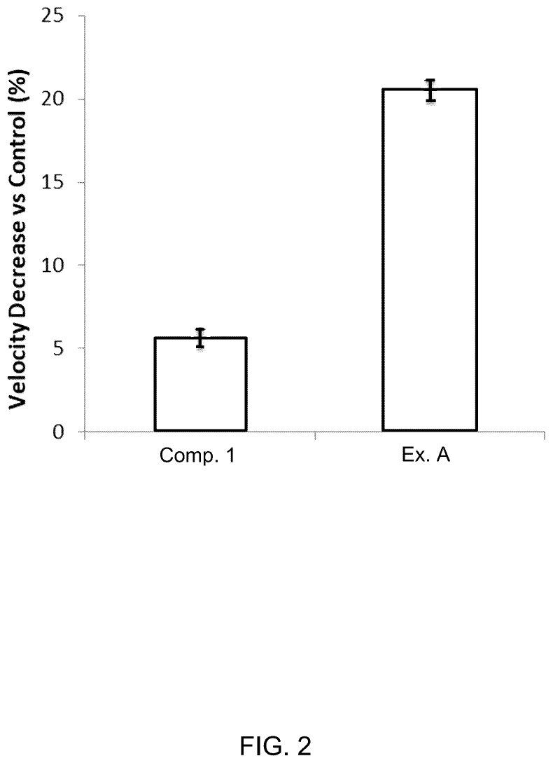

Example Composition A was evaluated to assess its anti-bleeding efficacy by way of a platelet aggregation assay. Using the platelet aggregation assay, the platelet area increasing ratio and platelet velocity decreasing ratio for Example Composition A and Comparative Composition 1 were evaluated. The platelet area increasing ratio and platelet velocity decreasing ratio are two key factors to indicate the hemostasis capability. A higher result means a better bleeding reduction efficacy. As seen in FIGS. 1 and 2, Example Composition A significantly increased anti-bleeding efficacy, especially as compared to Comparative Composition 1 in this in-vitro model. This suggests that Example Composition A has a faster gingival bleeding relief speed in clinical performance.

### Example 3

A clinical trial was conducted to evaluate the effectiveness of Example Composition A on gingival bleeding. Example Composition A was evaluated in comparison to a fluoride containing comparative composition (Comparative Composition 2) over 3 weeks of usage. The formulation of Comparative Composition 2 is shown in Table 2.

TABLE 2

| | Comparative Composition 2 |
|---|---|
| US INCI Compound Name | (wt. %) |
| Sorbitol | 14.5-16 |
| Carrageenan | 0.9-1.3 |
| Dicalcium Phosphate Dihydrate | 42-46 |
| Sodium Lauryl Sulfate | 2.1-2.7 |
| Cocamidopropyl Betaine | 0.1-0.4 |
| Tetrapotassium Pyrophosphate | 0.4-0.6 |
| Sodium Monofluorophosphate | 1-1.3 |
| Flavoring and Saccharin | 1.5 |
| Water | QS to 100 |

Specifically, a two-cell, randomized, double-blind, parallel-group clinical trial was conducted using healthy subjects. The subjects were randomly assigned to one of two growths, with one of the groups being instructed to use Example Composition A and the other group being instructed to use Comparative Composition 2. The subjects were evaluated on a bleeding index initially before the start of the trial (baseline evaluation), after 3 weeks of using either Example Composition A or Comparative Composition 2, after 3 months of using either Example Composition A or Comparative Composition 2, and after 6 months of using either Example Composition A or Comparative Composition 2. A dentist conducted a final oral examination of the subjects prior to discontinuation of the study. Analyses of Covariance (ANCOVA's) was employed to compare the effects of using either Example Composition A or Comparative composition 2 with respect to each of the clinical parameters at the evaluated time points in view of the baseline evaluation as the covariate.

The results of the clinical trial for this Example are summarized in Table 3. As seen in Table 3, Example Composition A significantly reduced gingival bleeding. As seen in Table 3, Example Composition A achieved a statistically significant grater level in gingival bleeding reduction and gingivitis reduction that Comparative Composition 2.

TABLE 3

| | | Percent | Percent | |
|---|---|---|---|---|
| Index | Treatment | Reduction | Difference | Significance |
| Gingival Bleeding | Example | 15.2% | 12.5% | p < 0.001 |
| | Composition A | | | |
| | Comparative | 2.4% | | |
| | Composition 2 | | | |
| Gingival Bleeding | Example | 3.8% | 7.3% | p < 0.001 |
| Sites (Scores of 1 | Composition A | | | |
| and 2) | Comparative | −3.8% | | |
| | Composition 2 | | | |
| Gingival Bleeding | Example | 33.3% | 21.1% | p < 0.001 |
| Severity (Scores | Composition A | | | |
| of 2 Only) | Comparative | 13.6% | | |
| | Composition 2 | | | |
| Number of | Example | 4.3% | 7.6% | p < 0.001 |
| Bleeding Sites | Composition A | | | |
| | Comparative | −3.6% | | |
| | Composition 2 | | | |
| Gingival | Example | 12.4% | 9.8% | p < 0.001 |
| | Composition A | | | |
| | Comparative | 3.0% | | |
| | Composition 2 | | | |
| Gingival Severity | Example | 10.4% | 8.5% | p < 0.001 |
| | Composition A | | | |
| | Comparative | 2.1% | | |
| | Composition 2 | | | |
| Gingival | Example | 13.8% | 11.2% | p < 0.001 |
| Interproximal | Composition A | | | |
| | Comparative | 2.9% | | |
| | Composition 2 | | | |

What is claimed is:

1. An oral care composition comprising:
oleanolic acid;
eugenol; and
a zinc source,
wherein the oleanolic acid and the eugenol are present in a weight ratio of the oleanolic acid to the eugenol of about 2:1 to about 3:1.

2. The oral care composition according to claim 1, wherein the oleanolic acid is present in an amount from about 0.01 to about 3 wt. %, based on the total weight of the oral care composition, and the eugenol is present in an amount from about 0.01 to about 1.5 wt. %, based on the total weight of the oral care composition.

3. The oral care composition according to claim 1, wherein the zinc source is selected from zinc citrate, zinc oxide, zinc phosphate, zinc lactate, zinc sulfate, zinc silicate, zinc gluconate, and a combination of two or more thereof.

4. The oral care composition according to claim 1, wherein the oral care composition comprises a zinc system comprising at least two zinc sources selected from zinc citrate, zinc oxide, and zinc phosphate.

5. The oral care composition according to claim 1 further comprising:
a basic amino acid in an amount from about 0.1 to about 10 wt. %, based on the total weight of the oral care composition.

6. The oral care composition according to claim 5, wherein the basic amino acid comprises arginine, lysine, histidine, or a combination thereof.

7. The oral care composition according to claim 1 further comprising:
one or more polyol in an amount from about 15 to about 50 wt. %, based on the total weight of the oral care composition.

8. The oral care composition according to claim 7, wherein the polyol comprises glycerin, inositol, maltitol, mannitol, sorbitol, xylitol, propylene glycol, polypropylene glycol (PPG), polyethylene glycol (PEG), a block copolymer of PPG and PEG, a saccharide, or a combination of two or more thereof.

9. The oral care composition according to claim 7, wherein the one or more polyol comprises a polyol system comprising glycerin and sorbitol.

10. The oral care composition according to claim 9, wherein the glycerin and sorbitol are present in a weight ratio of glycerin to sorbitol of about 1:1 to about 3:1.

11. The oral care composition according to claim 1 further comprising:
an abrasive present in an amount from about 1 to about 35 wt. %, based on the total weight of the oral care composition.

12. The oral care composition according to claim 11, wherein the abrasive is selected from the group consisting of silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof.

13. The oral care composition according to claim 1 further comprising:
a fluoride source, optionally, in an amount from about 0.1 to about 7 wt. %, based on the total weight of the oral care composition, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

14. The oral care composition according to claim 1 further comprising:
an anti-calculus agent present in an amount from about 0.5 to about 10 wt. %, based on the total weight of the oral care composition.

15. The oral care composition according to claim 14, wherein the anti-calculus agent is selected from tetrasodium pyrophosphate, ammonium tripolyphosphate, hexametaphosphate salt, polycarboxylate polymers, polyvinyl methyl ether/maleic anhydride (PVME/MA) copolymers, and a combination of two or more thereof.

16. The oral care composition according to claim 1 further comprising:
a thickening agent present in an amount from about 1 to about 8 wt. %, based on the total weight of the oral care composition.

17. The oral care composition according to claim 16, wherein the thickening system comprises a thickening silica.

18. An oral care composition comprising:
oleanolic acid;
eugenol;
a zinc source; and
a basic amino acid.

19. The oral care composition according to claim 18, wherein the basic amino acid comprises arginine, lysine, or a combination thereof.

20. An oral care composition comprising:
oleanolic acid;
eugenol, wherein the oleanolic acid and the eugenol are present in a weight ratio of the oleanolic acid to the eugenol of about 2:1 to about 3:1; and
a zinc system comprising at least two zinc sources selected from zinc citrate, zinc oxide, and zinc phosphate.
